(19) United States
(12) Patent Application Publication
(10) Pub. No.: US 2021/0012162 A1
(43) Pub. Date: Jan. 14, 2021

(54) 3D IMAGE SYNTHESIS SYSTEM AND METHODS

(71) Applicant: Shenzhen Malong Technologies Co., Ltd., Shenzhen (CN)

(72) Inventors: Yawen HUANG, Shenzhen (CN); Weilin HUANG, Shenzhen (CN); Matthew Robert SCOTT, Shenzhen (CN)

(21) Appl. No.: 16/900,870

(22) Filed: Jun. 13, 2020

Related U.S. Application Data
(60) Provisional application No. 62/871,724, filed on Jul. 9, 2019.

(51) Int. Cl. G06K 9/62; G06K 9/00; G16H 30/40; G06N 3/02

(52) U.S. Cl. CPC G06K 9/6262; G06K 9/6256; G06N 3/02; G06K 9/00201; G16H 30/40; G06K 9/6253

(57) ABSTRACT

Aspects of the technology described herein provide a system for improved synthesis of a target domain image from a source domain image. A generator that performs the synthesis is formed based on texture propagation from the first domain to the second domain by making use of a bidirectional generative adversarial network. A framework is provided for training that includes texture propagation with a shape prior constraint.

### CROSS-REFERENCE TO RELATED APPLICATIONS

This application claims the benefit of U.S. Provisional Application No. 62/871,724, filed Jul. 9, 2019, entitled “3D Image Synthesis System and Methods,” the benefit of priority of which is hereby claimed, and which is incorporated by reference herein in its entirety.

### BACKGROUND

There are many cases in which 2-D images, or frames, are applied in art, medicine, manufacturing, computer-aided design (CAD), animation, motion pictures, and computer-aided simulation, etc. There are many instances in which a user may wish to synthesize a frame from known data. To name a few examples: a data frame in a sequence of frames may be missing, corrupted, or inadvertently deleted. A subject may move during data collection, resulting in one or more distorted frames. A data collection system may operate in only one mode at a time, providing data in a single-mode, when additional modes of data are desired. An operator may wish to estimate what another mode of data collection would have looked like, given an input image.

To consider one such example in more detail: a clinician, while performing a recent, annual T-2 weighted MRI scan for a patient that presented with epileptic seizures, notices an indication of a tumor. When a T-1 weighted scan is also performed, the tumor's current morphology is revealed. However, the clinician would like to know about the tumor's growth rate, from a prior time, unfortunately, no T-1 weighted scan is available. The clinician can access the T-2 weighted MRI image depicting the same area from the prior year. The clinician would like to estimate morphology from the prior year. The clinician would like to estimate the T-1 weighted MRI data would have looked like, given the T-2 weighted MRI data that was collected in the prior year. Accordingly, there is a need in this and similar circumstances for a method that synthesizes, even with clinical accuracy, an image frame such as a T-2 weighted MRI image from an available image frame such as a T-1 weighted MRI image.

### BRIEF DESCRIPTION OF THE DRAWINGS

Aspects of the technology described in the present application are described in detail below with reference to the attached drawing figures, wherein:

FIG. 1 is an illustration of a user interface operative to control a system to generate a target domain image from a source domain image;

FIG. 2 is a diagram of a computer system configured to generate a target domain image from a source domain image, and to train a synthesizer;

FIG. 3 is a logical flow diagram illustrating a method of training a generator to produce a target domain image from a source domain image using domain matching, texture propagation, and a prior shape constraint;

FIG. 4 is a system diagram depicting exemplary components used in training a bidirectional generative adversarial network including a generator G that generates a target image from a source image using domain matching, texture propagation, and shape prior constraints;

FIG. 5 is a block diagram of an iterative method of training a generative adversarial network using entropy loss aggregated from one or more sources of loss;

FIG. 6 is a block diagram illustrating a method of defining classes for an application that makes use of a shape prior constraint; and

FIG. 7 depicts an embodiment of an illustrative computer operating environment suitable for practicing embodiments of the present disclosure.

### DETAILED DESCRIPTION

The subject matter of the present disclosure is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

As one skilled in the art will appreciate, embodiments of this disclosure may be embodied as, among other things: a method, system, or set of instructions embodied on one or more computer-readable media. Accordingly, the embodiments may take the form of a hardware embodiment, a software embodiment, or an embodiment combining software and hardware. In one embodiment, the present technology takes the form of a computer-program product that includes computer-usable instructions embodied on one or more computer-readable media.

This disclosure is related to the use of an Artificial Neural Network (ANN) such as a Convolutional Neural Network (CNN), or a Generative Adversarial Network (GAN) to perform image synthesis. An ANN is a computer processing module in hardware or software that is inspired by elements similar to those found in a biological neuron. For example, a variable input vector of length N scalar elements v₁, v₂, . . . vₙ are weighted by corresponding weights wᵢ, and to an additional bias b₀, and passed through hard or soft non-linearity function h( ) to produce an output. In an embodiment, the nonlinearity is for example a sign function, a tan h function, a function that limits the maximum or minimum value to a programmable threshold output level, or a ReLU function. An ANN may produce output equal to h(v₁*w₁+v₂*w₂+ . . . +vₙ*wₙ+b₀). Such networks “learn” based on the inputs and on a weight adjustment method. Weights may be adjusted iteratively based on evaluating the ANN over a data set while modifying the weights in accord with a learning object. Generally, an ANN with a plurality of layers is known as a deep network.

A Convolutional Layer is a layer of processing in a convolutional neural net hierarchy. A layer is a set of adjacent ANNs that have a small and adjacent receptive field. Typically a CNN has several defined layers. In an embodiment, a layer attribute such as identity, interconnection definitions, layer characteristics, layer type, number of layers may be set within a CNN component. The number of layers, for example, can be chosen to be 6, 16, 19, 38, or another suitable number.

A CNN is an ANN that performs operations using convolution operations, typically for image data. CNN may have several layers of networks that are stacked to reflect higher level neuron processing. A layer in a CNN may be fully connected or partially connected to a succeeding layer. One or more layers may be skipped in providing a layer output to a higher layer. The convolutions may be performed with the same resolution as the input, or a data reduction may occur through the use of a stride different from 1. The output of a layer may be reduced in resolution through a pooling layer. A CNN may be composed of several adjacent neurons, which process inputs in a receptive field that is much smaller than the entire image. Examples of CNN components include ZF Net, AlexNet, GoogLeNet, LeNet, VGGNet, VGG, ResNet, DenseNet, etc.

A Corpus is a collection of samples of data of the same kind, wherein each sample has two-dimensional (picture), three dimensional (voxel), or N-dimensional extent. A collection may be formed for example from similar types of samples, that have a common set of attributes. Attributes of a sample may include the portion of anatomy (brain, head, heart, spine, neck, etc.), the mode or modality of the collection (FLAIR, T1-Weighted, T2-Weighted, PD-weighted, structural MRI, CT), the underlying technology (Magnetic Resonance Imaging (MRI), photograph, X-ray, Computer-Aided Tomography (CAT), Graphic Sequence, animation frame, game frame, simulation frame, CAD frame, etc.). Attributes further may include the date, subject condition, subject age, subject gender, technician collecting data, etc.

An entropy loss term is a term quantifying an amount of disorder. As an objective function argument, an entropy loss can be defined in various ways to meet an objective criterion that quantifies distance from an objective.

A GAN is a network of ANN elements that includes at least a generator network such as g( ) and a discriminator network such as dg( ) The generator network maps an input source domain sample x to form a synthesized output ŷ that approximates a target domain sample y. The discriminator network dg( ) judges whether a mapped output is real or fake. The generative adversarial network is then optimized by adjusting weights within both dg( ) and g( ) while maximizing the entropy at the output of the discriminator dg( ) but minimizing the entropy at the output of the generator g( ).

A bidirectional GAN may have dual-arranged synthesizers, that is, in addition to a first generator g( ) and a first discriminator dg( ) also includes a second generative network f( ) that operates in the reverse direction, approximating an inverse to the first generator g( ) by mapping an output target domain sample y to form a synthesized input \hat{x} that approximates a source domain sample x, A bidirectional GAN may also include a second discriminator df( ) that judges whether a pseudo-input is real or fake. In a bidirectional GAN, the mappings can be composed to form a pseudo sample that is based on both composed mappings. A pseudo-input x′ is given by f(g(x)). A pseudo-output y′ is given by g(f(y)).

A norm is a generally positive length measure over a vector space. In an embodiment, a norm comprises a semi-norm. A 2-norm is the square root of the sum of the squares of the vector elements. A 1-norm is the sum of the absolute values of the vector elements. A p-norm is a quantity raised to the 1/p power that includes a sum of the absolute values of the vector elements, wherein each absolute value of an element is raised to the p power. An infinity norm is the max over the vector elements of the absolute value of each vector element.

A Residual Neural Network (RNN) is an ANN that feeds the neural output to a layer beyond the adjacent layer, skipping one or more intervening layers, so that the receiving layer forms a result that includes a neural input from a non-adjacent preceding layer

A Segmentor is a network that segments the pixels of an image into a number of segment classes, e.g. class c1, c2, c3, . . . . The output of a segmentor operating on a pixel may be a set of class labels or a probability vector that reflects a probability that the pixel is a member of each of the segment classes.

Computer-readable media can be any available media that can be accessed by a computing device and includes both volatile and nonvolatile media, removable and non-removable media. By way of example, and not limitation, computer-readable media comprises media implemented in any method or technology for storing information, including computer-storage media and communications media. Computer storage media includes both volatile and nonvolatile, removable and non-removable media implemented in any method or non-transitory technology for storage of information such as computer-readable instructions, data structures, program modules, or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by a computing device.

Computer storage media does not comprise signals per se. Communication media typically embodies computer-readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared, and other wireless media. Combinations of any of the above should also be included within the scope of computer-readable media.

In some embodiments, the disclosed Multi-Task Coherent Modality Transferable GAN (MCMT-GAN) is used for volumetric neuro-image processing in an unsupervised manner. The solution includes a generic way of learning a dual mapping between source and target domains while considering both visually high-fidelity synthesis and later segmentation practicability. Through combining the bidirectional adversarial loss, cycle-consistency loss, domain adapted loss, and manifold regularization in a volumetric space, MCMT-GAN is robust for medical image synthesis under multiple conditions. In addition to generating the desirable modality data, this solution complements discriminators collaborative working with segmentors which balances synthesis fidelity and segmentation performance. Experiments evaluated on several cross-modality syntheses show that the disclosed solution produces visually impressive results, and the generations can substitute real acquisitions for clinical post-processing, and also exceeds the state-of-the-art methods.

Medical imaging enjoys a multitude of imaging modalities such as Computed Tomography (CT), Magnetic Resonance Imaging (MRI) and Positron-Emission Tomography (PET), and opens up the opportunity to gain insights into diverse tissues' characteristics via different physical acquisition principles or parameters. The simultaneous availability of multi-modal imaging has benefited a wide range of brain image analysis tasks, for example, providing complementary information to discriminate specific tissues, anatomies, and pathologies in image segmentation, or improved cross-modal image registration under the great variability of tissue or organ appearance

Despite that neuro-imaging such as MM can produce highly detailed images with great soft-tissue contrast, one practical problem remains largely unsolved: imaging has notoriously long acquisition times, which hampers multi-protocol MRI acquisitions at high-resolution. Additional complications arise when imaging patients with some specific medical conditions; for instance, Alzheimer's disease and Parkinson's disease, require that the acquisitions are collected in even short times resulting in very low-quality images.

This disclosure includes a cross-modality image synthesis solution, which is related to texture transfer, also known as image-to-image translation or style transfer. In some embodiments, it is to render a source modality image into a target one, e.g., synthesizing a CT as an MRI, or a PD-w MRI as a T2-w MRI, or a structural MRI as the diffusion tensor image. Due to a lack of paired information, the unsupervised cross-modality synthesis problem is much harder but more applicable in clinical routines and post-processing (e.g., segmentation or registration). When the underlying relationship between different modalities is known a priori, the design of cross-modality image synthesis can ideally be specifically task-driven, such that the synthesized results are tailored to the information to determine them within the respective reliability standards.

Moreover, the generated images may present additional information to improve the task performance interactively. In an embodiment, it is to tackle the problem of segmentation-driven unsupervised cross-modality synthesis by exploring the deep architecture for volumetric neuroimage processing. In an embodiment, the disclosed MCMT-GAN can learn the cross-modality transformation in an unsupervised manner for 3D medical images. In the absence of any paired data, a jointly-adapted bidirectional loss may consist of three sub-components: conditional dual mapping, volumetric cycle-consistency, and domain adaption. The presented loss function utilizes the deep features to penalize domain discrepancy while ensuring the bidirectional mappings modeled in a closed-loop are using the criterion of cycle-consistency to improve data variations within multiple conditions. Moreover, this technical solution incorporates the manifold structure to enrich the feature representation with more tissue discriminative and fine-grained capability. To achieve not only visually-realistic synthesis but also task-practicability, the solution may complement the bidirectional discriminators with segmentors that balances both synthesis fidelity and segmentation performance. Rather than conditioned on the segmentation descriptions, the proposed fault-aware discriminator is capable to improve the performance of segmentation while preserving strong visual effects.

In an embodiment, this solution solves the cross-modality synthesis problem driven by segmentation in volumetric neuroimaging under an unsupervised setting. In an embodiment, through combining both adversarial losses of dual mappings, the cycle-consistency loss in volumetric space, and domain adapted loss, this solution uses a jointly-adapted bidirectional loss for solving the problem of medical images under multiple conditions. In an embodiment, a well-specified manifold regularizer is devised to focus on exploring the geometric structure of the data distribution underlying the respective domains. In an embodiment, this solution uses a fault-aware discriminator to improve the performance of segmentation from the synthesized results using a combination of adversarial losses and manifold regularization conditioned on the segmentation performance.

Generally, prior methods have suffered from a limitation associated with supervised learning. The analysis of medical images is always required for both diagnostic and therapeutic medicine in many clinical tasks. Among applications for investigating the properties of tissue organizations, image segmentation is a major task. It faces a problem when acquisitions have a variability of tissue appearance obtained from different physical principles. Therefore, efforts to tackle it can focus on the cross-modality synthesis.

Generative Adversarial Networks (GANs) are composed of two models: generator G and discriminator D. G is trained to imitate the real image by mapping a latent random vector z sampled from the uniform noise distribution [symbol] to the real data distribution p_{data}. D is optimized to distinguish whether an image is the generated counterpart G(z)˜p_{g} or the real one x˜p_{data}. Concretely, given the vectorized image x, G and D are defined to solve the following adversarial minimax objective on V (D, G):

$${min}_{G} {max}_{D} V ( D , G ) = {𝔼}_{x ∼ {p}_{data}} [ log D ( x ) ] + {𝔼}_{z ∼ {p}_{z}} [ log ( 1 - D ( G ( z ) ) ) ]$$

The above equation can be solved in an alternative manner over generator G and discriminator D. That is, by fixing the parameters of G, this solution can optimize D and vice versa. There exists global optimality when p_{g}=p_{data} with a mild condition that G and D have enough capacity to make p_{g} converges to p_{data}· [symbol] _{cyc}(G, F) is combined with the dual mapping functions G:X→Y and F:Y→X into the GANs' objective for unpaired cross-modality synthesis. X and Y are two sets of training samples as X=(x₁, x₂, . . . xₛ) and Y=(y₁, y₂, . . . yₜ).

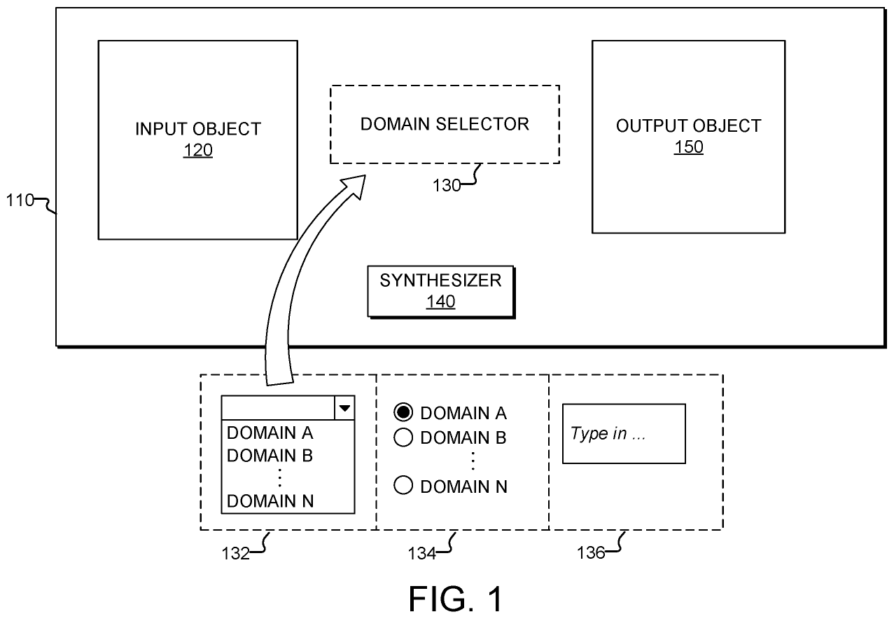

Turning now to FIG. 1, there is depicted therein a user interface operative to control a system to generate a target domain image from a source domain image. Computer display screen 110 presents graphical display area (GDA) 120 showing in input object, a domain selector control 130, an output object display area (OBDA) 150, and a synthesizer display area 140 that describes an underlying synthesizer 420.

In an embodiment, GDA 120 serves as a graphical control for inputting an object from a source domain, and indicating the characteristics of the input object such as the associated source domain of the input object. In an embodiment, a header or file name extension of the input object indicates the type of source domain represented by the input object. An input object such as a source domain image is identified to synthesizer 420 as the input image from a source domain, e.g. Domain A. A user selects an object, e.g. by use of a computer pointing device, selecting an object from a source domain and dragging the object over GDA 120, thus informing synthesizer 420 of a desire to generate an output object to be displayed in OBDA 150.

Synthesizer display area 140 displays a description of a pre-determined synthesizer 420 that is capable of generating an output target domain image from an input source domain image. In an embodiment, a description includes an attribute of synthesizer 420 such as a title (e.g. BrainGAN23), a model developer name, a list of supported modes, an image context, several segment classes, a description of training data used, a date, etc. In an embodiment, synthesizer 420 has a predetermined output domain setting, such as Domain B, and so synthesizer 420 generates an output object in accord with the output domain setting, such as Domain B, and displays the output object in OBDA 150.

In an embodiment, domain selector control 130 comprises one or more of a list box 132, a radio button selector 134, and a domain input field 136. A user activates list box 132 by selecting the down-arrow list control and scrolling through several supported domains to select a particular member of the list such as “domain B” for output. List box 132 illustrates the availability of multiple different Domains: domain A, domain B, . . . domain N. Likewise radio button selector 134 allows the user to select a single output domain such as Domain A shown. Domain input field 136 allows the user to type in a description or designator for the desired output domain.

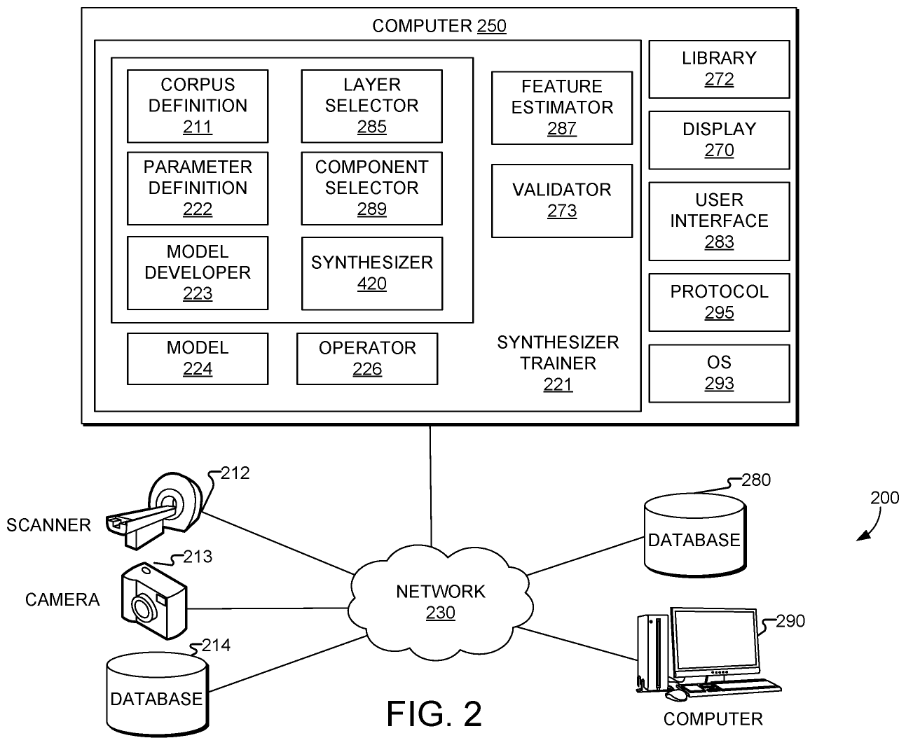

Turning now to FIG. 2 there is shown a diagram of a computer system operative to control a system to generate a target domain image from a source domain image. An operator program 226 runs in the memory of computer 250, responding to and invoking other programs that run in the memory of computer 250, in cooperation with operating system 293. Images are collected for synthesizer 420, e.g. by the operation of a sensor such as scanner 212 or camera 213, and images are stored, for example in local database 214 and/or remote database 280. An operator program 226 can browse and select images that are located on computer 250, on the local database 214 and the remote database 280 by making use of operating system 293, and protocol module 295. In an embodiment, network 230 comprises a Local Area Network (LAN) that connects database 214, camera 213, scanner 212 to computer 250, and a Wide Area Network (WAN) that connects computer 250 to computer 290 and database 280. In an embodiment, network 230 comprises a bus 710 that connects scanner 212 camera 213 and database 214 to computer 250, e.g. through I/O ports 750.

Operator program 226 functions to present to the user a display screen 110 using display module 270, and also to receive user indications from the user through user interface module 283. Operator program 226 retrieves the input source image from database 214, and displays a representation of the input source image in GDA 120. Operator program 226 reads the attributes of model 224 and presents a description of the attributes of model 224 to the user in synthesizer display area 140. Operator program 226 receives a user indication such as a domain selection received in domain selector control 130, indicating a desire to generate an output image in a determined target domain. Operator program 226 selects an appropriate model such as model 224 from a library 272 and loads the model 224 into synthesizer 420. Appropriate model selection considers one or more of a source image domain attribute, target domain image attribute, user indication of the desired model, a recently used model, user feedback indicating acceptable or unacceptable past behavior by a model, etc. In an embodiment, operator program 226 selects a performing model from library 272 that meets one or more user indicated aspects of a model. Operator program 226 uses the synthesizer 420 to synthesize an output target image, which the operator program 226 then displays in OBDA 150.

Library 272 generally includes all data and objects used or referenced by system 200. Portions of library 272 are residents for example in database 214 or database 280. Library 272 includes, for example, models, model definitions, model status, model context, supported model modes, and supported model classes, software, software development SDK's, software APIs, CNN libraries, CNN development software, etc.

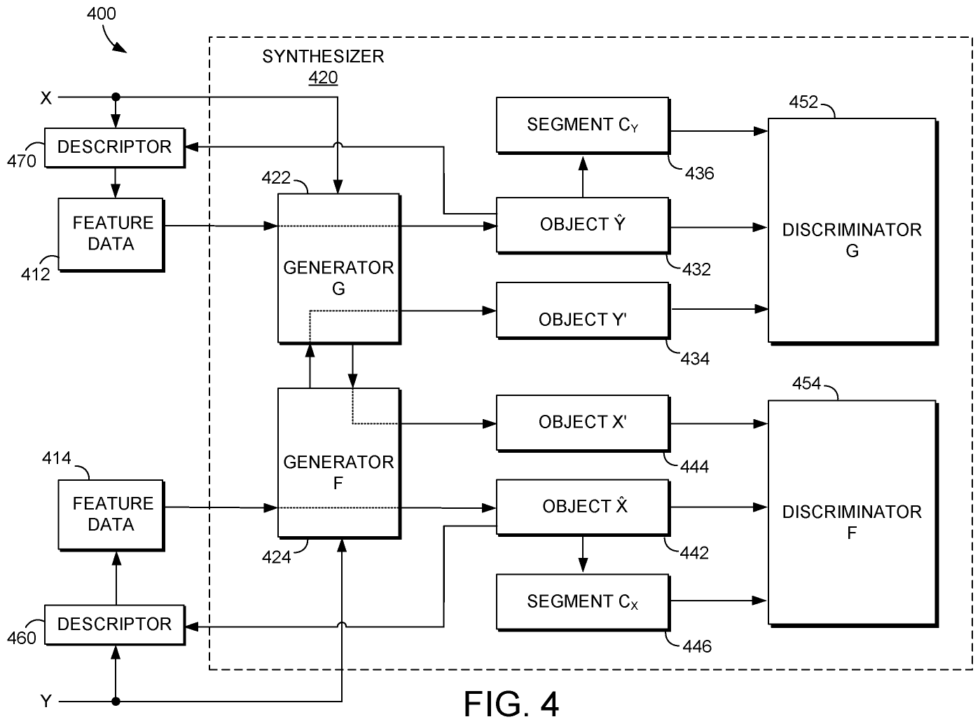

Synthesizer 420, when loaded with an appropriate model 224 is configured to synthesize a target domain image from a source domain image. Model 224 generally includes attributes that define an operational synthesizer 420 that includes weights and biases of one or more ANNs defining one or more of a generator 422, a generator 424, a segmentor 436, and a segmentor 446. The weights and biases of an ANN are stored in their usable form, e.g. through prior configuration and training by the operation of synthesizer trainer 221. Synthesizer 420 includes generator 422 and generator 424 that have been trained in a bidirectional generative adversarial network configuration. Since the training of generator 422 and generator 424 are simultaneously trained within a bidirectional generative adversarial network as described herein concerning FIG. 4, generator 424 will be an approximate inverse of generator 422. Generator 422 is based on training with texture propagation when synthesizer 420 has been loaded with a model 224 that provides weights and biases to generator 422 that has been trained using a method that influences a generator to perform texture propagation. Generator 422 is based on training with domain matching when synthesizer 420 has been loaded with a model 224 that provides weights and biases to generator 422 that has been trained using a method that influences a generator to perform domain matching. Generator 422 is based on training with a segmentation constraint when synthesizer 420 has been loaded with a model 224 that provides weights and biases to generator 422 that has been trained using a method that influences a generator to operate with a segmentation constraint.

Synthesizer trainer 221 is configured to define and configure synthesizer 420 for training. Synthesizer trainer 221, using component selector 289, selects individual components used in training such as descriptor 470, descriptor 460, generator 422, generator 424, segmentor 436, segmentor 446, discriminator 452 and discriminator 454. Component selector 289 presents the user with a component identity option for a synthesizer or trainer component, and receives an indication to define a component to have a particular form, such as a specific CNN to be used as a component. Layer selector 285 defines a layer to be used by the synthesizer trainer 221. Based on user selection, the layer selected is used within a user-selected context such as a layer to output to another layer, or a layer attribute definition, or a particular layer to be used for feature extraction, or to be used in a loss calculation. Parameter definition module 222 defines and stores parameter settings that are used for training based on user input. Examples of parameters include the relative weighting of loss calculations in a combined loss calculation, the stride number in a convolutional layer, the number of layers in a CNN, the type of layer in a CNN, the type of layer to be used (e.g. partially connected, fully connected, pooling, etc.), the size of discriminator, the kernel size to be used, the activation method, learning rate, weight modification method, the solver to be used for training, the mini-batch size, etc. Model developer module 223 presents to the user the content of a model definition, describing the corpus defined by corpus definition module 211, the parameters defined by parameter definition module 222, the layers selected for use by layer selector 285, the components selected for use by component selector 289, the estimator selected by feature estimator 287. Model developer module 223 also displays the status of models as partially defined, completely defined, trained, validated, history of use, history of success, history of failure, etc.

In an embodiment, synthesizer 420 makes use of generator 422 or 424 with a particular operational configuration. In an embodiment, a generator consists of 3 convolutional layers with strides 1, 2, and 2 as the front-end, 6 residual blocks, 2 fractionally-strided convolutions with the same stride 1/2, and 1 convolutional layer as the back-end with stride 1. The general convolutional layers with spatial batch normalizations and ReLU nonlinearity are added in between (i.e., formed as convolution-BatchNorm-ReLU), while the output layer applies the tan h activation at the end. The 6 residual blocks each includes 2 convolutional layers with the fixed 128 filters on both layers. Particularly, in some embodiments, this solution uses the 7×7×7 volumetric kernels for the first and last layers while using the 3×3×3 volumetric kernels for the remaining layers.

In an embodiment, synthesizer 420 makes use of discriminator 452 or 454 that has a particular operational configuration. In an embodiment, for the discriminative network, the solution may adopt the Marko-vian PatchGAN but mirrors the generator in the volumetric space. Instead of modeling the full image-sized discriminator, Markovian PatchGAN effectively models an image as a Markov random field in local image patches for distinguishing whether the selected size of patch in an image is real or fake. Such a configuration is effective especially in the very large-sized images and 3D volumes since it contains parameters fewer than others. The solution may fix the patch size as 70×70×70 in the overlapped manner and use the stack of convolution-BatchNorm-Leaky ReLU layers (i.e., instead of using ReLU, Leaky ReLU activation is applied here) to train the discriminative network. In an embodiment, the discriminator is run convolutionally across the volumes and finally averaging all responses to give the ultimate results.

In an embodiment, synthesizer 420 makes use of segmentor 436 or segmentor 446 that has a particular operational configuration. In an embodiment, a segmentor is implemented as a deconvolution operation. In an embodiment, a layer skip architecture is employed for the segmentor. In an embodiment, all layers of the segmentor are adapted. In an embodiment, a segmentor is trained on a per-pixel basis. In an embodiment, a segmentor is validated with standard metrics.

Turning to FIG. 4, a system 400 includes exemplary components used in training a bidirectional GAN including generator 422 (G) that generates a target image from a source image using domain matching, texture propagation, and segmentation constraints. Generator 422 receives a source sample X and generates a synthesized target Y represented in object 432 that estimates a target domain sample. Generator 424 receives a target sample Y and generates a synthesized source \hat{X} represented in object 442 that estimates a source domain sample. A pseudo-source X′ represented by object 444 is formed by generator 424 operating on the output of generator 422. A pseudo-target Y′ represented by object 434 is formed by generator 424 operating on the output of generator 422.

Discriminator 452 operates with the knowledge of segment membership taken from segmentor 436 to determine if pseudo target object 434 and synthesized object 432 are real or fake. Discriminator 454 operates with knowledge of segment membership taken from segmentor 446 to determine if synthesized object 442 and pseudo source object 444 are real or fake. Descriptor 470 estimates features over samples X in the source domain corpus and stores the feature estimates in feature data store 412. A descriptor network such as 470 also estimates features by operating on the synthesized target Ŷ and stores the feature estimates in feature data store 412. Descriptor 460 estimates features over samples Y in the target domain corpus and stores feature estimates in feature data store 414. A descriptor network such as descriptor 460 also estimates features by operating on synthesized source \hat{X} and stores the feature estimates in feature data store 414.

Synthesizer trainer 221 uses descriptor 470 to form generator 422 based on texture propagation. Feature data at feature data store 412 is used in the formation of generator 422. The features stored in feature data store 412 influence the development of generator 422 and/or generator 424. Synthesizer trainer 221 uses descriptor 460 to form generator 424 based on texture propagation. Feature data at feature data store 414 is used in the formation of generator 424. The features stored in feature data store 414 influence the development of generator 424 and/or generator 422. In an embodiment, synthesizer trainer 221 effects feature influence by using a texture propagation entropy loss term as grounds to modify the values of the weights and biases present in an ANN contained within a generator, thus propagating the features from a source domain to a target domain in the creation of a generator. In an embodiment, descriptor 470 and descriptor 460 are implemented as CNN deep networks, such as VGG. Descriptors such as descriptor 470 and descriptor 460 comprise feature maps that are processed during training to preserve local textural details at convolutional layers. For example, by training with a texture propagation objective, generator 422 causes a source image to propagate textural details from a source image to a target image, thus achieving texture propagation.

In an embodiment, feature maps such as the feature maps of descriptor 460 or descriptor 470 are compared at a modeling layer L. In an embodiment, the feature maps at layer L are compared to other feature maps at layer L. In an embodiment, all feature maps at layer L and below are compared to all feature maps at layer L and below. The feature maps at layer L that model features of a target domain sample within descriptor 460 are compared to the feature maps at layer L that model features of a synthesized source domain sample (e.g. object 442), also within descriptor 460. The feature maps at layer L that model features of a source domain sample in descriptor 470 are compared to the feature maps at layer L that model a synthesized target domain sample (e.g. object 432), also within descriptor 470. An entropy loss term that quantifies an objective for optimization is calculated from the norm of the difference between the feature maps at layer L within Descriptor 470, added to a norm of the difference between the feature maps at layer L within Descriptor 460. In an embodiment, the 1-norm is used for such an entropy loss term.

Synthesizer trainer 221 uses segmentor 436 to effect a segmentation constraint in the development of the weights and biases of generator 422. In an embodiment, segmentor 436 extracts shape information from target domain samples such as object 432 and object 434. Likewise, segmentor 446 extracts shape information from source domain samples such as object 442 and segmentor 446 In an embodiment, synthesizer trainer 221 operates discriminator 452 and generator 422 by measuring segmented versions of Y over the corpus of target domain samples and segmented versions of the synthesized target Ŷ over the source domain corpus. In an embodiment, synthesizer trainer 221 operates discriminator 454 and generator 424 by measuring segmented versions of X over the corpus of source domain samples and segmented versions of synthesized source domain sample \hat{X} over the target domain corpus. In an embodiment, synthesizer trainer 221 effects segmentation constraint by using a segmentation entropy loss term as grounds to modify the values of the weights and biases present in an ANN contained within generator 422 and generator 424, thus forming generator 422 and generator 424 with, a segmentation constraint. In an embodiment, segmentor 436 and segmentor 446 forms a segmentation cross entropy term that calculates cross entropy loss across a set of classes. In an embodiment, the set of classes includes Cerebrospinal Fluid, Gray Matter, and White Matter.

In an embodiment, synthesizer 420 comprises dual arranged generator 422 and generator 424. Generator 422 receives geometric structure features over a pathway from feature data store 412. Generator 422 then generates a synthesized target object 432 based on the geometric structure features received. For example, the weights and biases of one or more layers of a CNN within generator 422 are modified based on training by synthesizer trainer 221 while reducing loss calculated within a feature manifold regularizer loss. Generator 424 likewise receives geometric structure features over a pathway from feature data store 414. Generator 424 then generates a second synthesized source object 442 based on the received geometric structure features that are received. Likewise, the weights and biases of one or more layers of a CNN within generator 424 are modified based on training by synthesizer trainer 221 while reducing loss calculated within a feature manifold regularizer loss.

Synthesizer trainer 221 effects domain matching by comparing the high-level features from layers in a deep network in the source domain and target domains to rectify a mismatch between source and target domains. In an embodiment, the high-level features of descriptor 470 that pertain to the source domain are compared to the high-level features of descriptor 460 that pertain to the target domain to calculate the distance between kernel mean embeddings. In an embodiment, a Maximum Mean Discrepancy (MMD) criterion is integrated into an adversarial training objective. In an embodiment, an empirical estimation is adopted to form a loss term that compares the high-level features of the source domain to the high-level features of a target domain based on a Gaussian kernel with a bandwidth parameter. In an embodiment, the MMD criterion is only adopted for features in the two highest layers. In an embodiment, the MMD criterion is adopted only incorporating features for the three highest layers. In an embodiment, the MMD criterion is adopted for the feature of all layers but the three lowest layers. In an embodiment, the MMD criterion is adopted for the features of all layers but the two lowest layers. In an embodiment, the MMD criterion is adopted based on a predetermined set of layers based on data structure analysis. In an embodiment, a domain matching criterion reduces domain discrepancy. In an embodiment, a domain matching criterion matches all orders of statistics for the high-level features that can be matched by using a loss term that affects the gradient search of the generative network through backpropagation. In an embodiment, an MMD criterion is adopted for a predetermined set of M layers.

Returning to FIG. 2, validator 273 can validate model 224. Validator 273 reads the model definition from model 224 and invokes a corpus definition module 211 to select appropriate images to be used in validating model 224. In an embodiment, model 224 includes both forward and reverse mappings. In an embodiment, both a forward validation corpus and a reverse validation corpus are defined. In an embodiment, a validation corpus is defined for source domain samples that are independent of the training set and encompass a quantity of at least 10% of the training data set size. In an embodiment, validator 273 operates incrementally as each new sample is generated. Model statistics in model 224 are updated to include user quality feedback. Validator 273 defines evaluation criteria, and performs a validation over a corpus while tabulating results pertaining to the validation evaluation criteria. Validation results are presented to a user for approval. Once approved by a user, the corpus is then validated and placed into library 272 and labeled as a validated model for future use. In an embodiment, validator 273 uses evaluation criteria that comprise a score of results based on a user review of synthesized images. In an embodiment, validator 273 uses evaluation criteria that quantitatively evaluate synthesized images using PSNR or SSIM values.

Feature estimator 287 operates to estimate features of a source domain sample or a target domain sample. In an embodiment, features of a domain are determined by feature estimator 287 based on a selected CNN that is trained with a corpus of domain samples. In an embodiment, features are extracted by feature estimator 287 by using statistical feature extraction methods such as nonparametric feature extraction, or unsupervised clustering. In an embodiment, features are extracted by feature estimator 287 using a descriptor neural network such as descriptor 470 or descriptor 460. In an embodiment, feature estimator 287 estimates the features of a descriptor CNN over a corpus from a domain.

Protocol module 295 operates to perform link, network, transport, session, presentation, and application layer protocols. Using protocol module 295, computer program modules on computer 250 send and receive data from sensors such as scanner 212, camera 213, local database 214, and remote database 280, and with computer programs running on remote computer 290 through network 230.

Synthesizer trainer 221 can perform training over a corpus of source domain samples and a corpus of target domain samples. Corpus definition module 211 receives a user indication of the scope of training samples to define a corpus of source domain samples to be used in training. For example, a user selects samples of attributes of a domain such as 3D, brain, healthy, T1-weighted MRI, etc. Corpus definition module 211 stores these selected attributes. Corpus definition module 211 then searches database 214 or database 280 to find samples that meet the domain criteria supplied by the user, reflecting one or more of the selected attributes. The results of the search are presented to the user in descending order of level of matching the selected attributes, and the user indicates which samples are to be included in the training. Corpus definition module 211 similarly receives a target domain description from a user and based on user indication or approval defines a corpus of target domain samples. In an embodiment, a user selects an incremental estimation option, and a corpus of samples is incrementally increased by one sample as each new sample is supplied to the system, resulting in an incremental modification of the weights and biases of an ANN within synthesizer 420.

In an embodiment, instead of working with 2D image stacks of original volumetric neuroimaging, the solution may input 3D volumes (i.e., X and Y) directly to ensure the intrinsic sequential information between consecutive slices. Given training samples

$$χ = {\{ {X}_{i} \}}_{i = 1}^{S} ∈ {ℝ}^{m \times n \times t \times S}$$

in the source domain and

$$𝒴 = {\{ {Y}_{i} \}}_{i = 1}^{T} ∈ {ℝ}^{m \times n \times t \times T}$$

in the target domain, one may form a closed loop between the dual tasks, i.e. X↔ [symbol] without the supervision of paired examples. Here, m and n are the dimensions of the axial view of the volumetric image, t denotes the size of an image along the z-axis, while S and T are the numbers of elements in the source and target training sets respectively. As with the existing dual GANs' learning, the solution may construct two mappings: G:X→ [symbol] and F: [symbol] →X in the volumetric space, therefore the generation of G and F can be represented as Ŷ=G (X) and \hat{X}=F(Y) respectively. Two adversarial discriminators D_{G} and D_{F} are modeled to distinguish the fake products corresponding to G and F.

The difficulties of synthesis work vary with multiple conditions, e.g., the volumetric representations, an unsupervised setting, different imaging modalities, imaging angles, or even different systems from different manufacturers. To encourage the synthesized results leading to the improvement of segmentation, the solution may generate visually-realistic images conditioned on a segmentor. Therefore, in an embodiment, the solution may address the segmentation-driven cross-modality synthesis by (1) building two GANs in a dual manner with the bidirectional loss; (2) minimizing the misaligned representations; (3) preserving the geometric structure via manifold learning; (4) all while conditioning the whole model on performing the segmentation task.

In an embodiment, a system uses a bidirectional GAN. To translate an image Xᵢ in X to an image in [symbol] through applying GANs' model, the solution may learn a function G:X→ [symbol] with the expected output Ŷᵢ=G(Xᵢ). The generator G is then argued by a discriminator D_{G} giving the likelihood that the input image Xᵢ has been sampled from the target domain. Similarly, to map an image Y₁ in [symbol] to an image in X which can be the dual task of G by training an inverse generator F: [symbol] →X having \hat{X}ᵢ−F(Yᵢ) with the corresponding discriminator D_{F}.

In an embodiment, the adversarial losses of both mapping functions are jointly expressed in the volumetric space:

[symbol]

_{d}(D_{G},D_{F},G,F)= [symbol] _{Y˜Pdata(Y)}[log D_{G}(Y)]+ [symbol] _{X˜Pdata(X)}[log(1−D_{G}(G(X)))]+ [symbol] _{X˜Pdata(X)}[log D_{F}(X)]+ [symbol] _{Y˜Pdata(Y)}[log(1−D_{F}(F(Y)))].

where [symbol] _{d} is the dual loss. The above equation forms a simple closed loop between two losses which extends the volumetric GANs into a dual learning manner and joint representations into a unified framework. In the unsupervised dual learning problem, one typical property is to force both learnings from each other to produce the pseudo-input. This is done by generating X′ for task X→ [symbol] and Y′ for task [symbol] →X respectively, where X′=F(\hat{X})=F(G(X)) and Y′=G(\hat{X})=G(F(Y)). The solution may enforce a volumetric cycle-consistency with GANs' model using

[symbol]

_{c}(X, [symbol] , [symbol] ,F)= [symbol] _{X˜Pdata(X)}∥X−F(G(X))∥₁+ [symbol] _{Y˜Pdata(Y)}∥Y−G(F(Y))∥₁

where ∥·∥₁ means that it may adopt l₁ distance to quantitatively compare the input data and reconstructed pseudo.

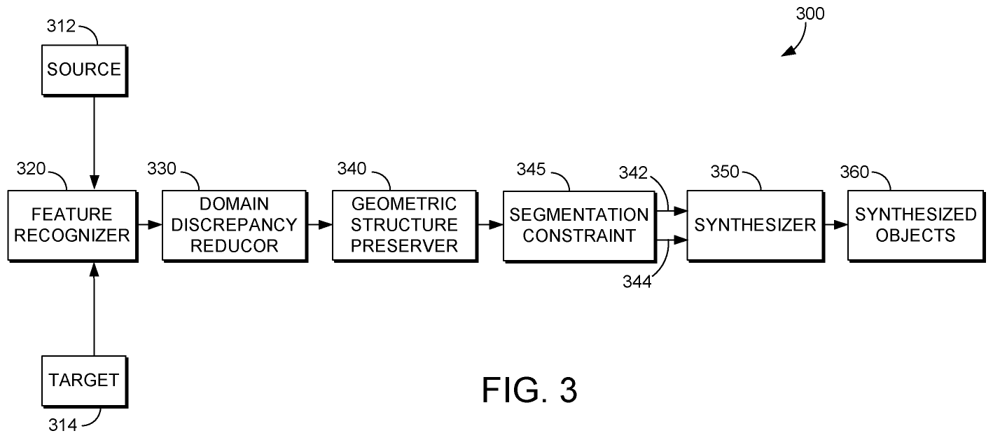

Turning to FIG. 3, a flow diagram illustrates a method 300 operable within synthesizer trainer 221 to train a generator to produce a target domain image from a source domain image using domain matching, texture propagation, and a segmentation constraint. At 312 the method receives a corpus of source samples. At 314 the method receives a corpus of target samples. Generally, method 300 operates through training that calculates an objective function and determines weights and biases of one or more ANNs using a bidirectional generative adversarial network, to produce an estimate at synthesizer 350 of a synthesizer 420 including generator 422 and generator 424 that can generate a synthesized object at block 360.

In an embodiment, the synthesizer 420 is formed by incorporating one or more of a domain discrepancy reduction computed at domain discrepancy reducer 330, a geometric structure preservation computed at geometric structure preserver 340 a segmentation constraint computed at block 345, a bidirectional constraint, and a cycle consistency constraint. In an embodiment, synthesizer 350 is formed by iteratively modifying weights and biases within an ANN performing generator 422 and within an ANN performing generator 424.

Feature recognizer 320 uses the corpus of source samples and the corpus of target samples to produce estimates of features in the source domain and to produce estimates of features in the target domain, that is, to identify the general features from one or more source objects in the source domain and one or more target objects in the target domain. In an embodiment, the features of the synthesized source domain and the features of the synthesized target domains are estimated at feature recognizer 320. In an embodiment, feature recognizer 320 recognize the features within descriptor 470 which as a CNN of six or more layers. In an embodiment, feature recognizer 320 recognize the features within a descriptor 460 which is a CNN of six or more layers. In an embodiment, feature recognizer 320 only selects the features of a predetermined number M of the layers for a constraint. In an embodiment, the M selected layers are the highest layers. In an embodiment, the M selected layers are the lowest layers. In an embodiment, the M selected layers are determined by statistical feature evaluation that determines the importance of the selected features for transfer. In an embodiment, the layers that are not among the M selected layers are identified as domain invariant. In an embodiment, the M selected layers are identified as domain invariant.

Domain discrepancy reducer 330 can reduce a discrepancy between the source and the target domains. A set of layers is determined in the source domain and in the target domain, e.g. a set of M layers in CNN descriptor 470, and in CNN descriptor 460. To reduce the domain discrepancy, domain discrepancy reducer 330 may apply an MMD criterion. In an embodiment, the method performed to reduce the discrepancy calculates a multi-Kernel MMD. In an embodiment, the distance between the mean embeddings of the features of the selected M layers is normed to form an entropy loss term. In an embodiment, domain discrepancy reducer 330 is coupled operatively to feature recognizer 320 to receive the features identified by a set of M layers in descriptor 470, and the features identified by a set of M layers in descriptor 460.

Geometric structure preserver 340 is employed to form estimates of generator 422 and generator 424, wherein generator 422 and generator 424 are configured in a bidirectional GAN such as synthesizer 420. In an embodiment, geometric structure preserver 340 processes the features in the M selected layers. In an embodiment, these M selected layers identify the features of a deep neural network. In an embodiment, the features of the source domain are modeled in descriptor 470. In an embodiment, the features of the target domain are modeled in descriptor 460. In an embodiment, graph learning is performed. In an embodiment, the geometric structure features of the first source object and a first target object are determined, or generated based on the selection of the M layers, that is, based on the identified domain invariant features. In an embodiment, the deep features are used for geometric structure measurement. For a set of features that correspond to each member of the source domain corpus and a set of features that correspond to each member of a target domain corpus, the solution may generate two q-nearest neighbor graphs with p vertices. The weight matrices for (1,1) in each domain are then defined to be 1 only when, for two deep features on the manifold with shortest geodesic distances, the features for a source sample i or the features for a target sample i are among the q-nearest neighbors of either the source features for a source sample j or the target features of target sample j. A manifold regularization entropy loss term is formed based on a sum over the p vertices of the graphs. In an embodiment, a loss term determines the geometric structure features of the domain-specific manifold information for the source domain, and determining the domain-specific manifold information of the target domain. In an embodiment, the loss term includes a normed distance between the features of sample i in the source domain and the features of sample j in the source domain when the samples are within the q-nearest neighborhood. In an embodiment, the loss term includes a normed distance between the features of sample i in the target domain and the features of sample j in the target domain when the features of sample i in the target domain and the features of sample j in the target domain are within the q-nearest neighborhood. In an embodiment, synthesizer 420, including generator 422 and generator 424, are trained using a loss term that reflects geometric structure over the features in the source and target domains. In an embodiment, a norm factor in a loss term is squared for calculating an entropy-related factor.

In an embodiment, the general features of a deep neural network such as descriptor 460 and descriptor 470 are used to identify features of a source object and features of a target object. In an embodiment, the general features are those features that are similar in the source and target domains. In an embodiment, the set of domain invariant features is identified by examining the behavior of the general features in a geometric structure based on the q-nearest neighbors of the general features. In an embodiment, the geometric structure features are calculated based on a selection of the M highest level layers.

In an embodiment, geometric structure preserver 340 is coupled to domain discrepancy reducer 330, to receive a set of M feature layers to be processed. Geometric structure preserver 340 then determines a geometric structure based on the identification by feature recognizer 320 of which are domain invariant. In an embodiment, geometric structure preserver, 340 operates on the M feature layers that are not domain invariant. Geometric structure preserver 340 determines geometric structure that operates a plurality geometric structure features of a training source object and based on a plurality of geometric structure features of the training target object. In an embodiment, the extent of a plurality is defined by the p vertices of a q-nearest neighborhood.

In an embodiment, the synthesizer 420 includes generator 422 and generator 424 that have been trained while reducing an entropy loss that includes a manifold regularization entropy loss term. Thus a synthesized object produced by generator 422 or generator 424 is based on training that involves the mapping of features between the source domain and the target domain. When the training also includes a segmentation loss term, the generator 422 and generator 424 are conditioned on a segmentation task.

At block 345 a segmentation constraint is applied in the formation of image generator 422. A segmentor 436 extracts segment information from synthesized object 432, and from target domain sample Y. A segmentor 446 extracts segment information from a source domain sample and from synthesized source object 442. In an embodiment, a first segmentor output such as a segment class, or a segment probability is output by segmentor 436 based on a target image sample. In an embodiment, a second segmentor output such as a segment class, or a segment probability is generated by segmentor 436 based on synthesized target object 432. In an embodiment, a segmentation loss term is formed based on a sum that includes an entropy derived from the first segmentor output and the entropy derived from the second segmentor output. In an embodiment, a third segmentor output such as a segment class, or a segment probability is output by segmentor 446 based on a source image sample. In an embodiment, a fourth segmentor output such as a segment class or a segment probability is output by segmentor 446 based on a synthesized input object 442. In an embodiment, a segmentation loss term is formed based on a sum that includes an entropy derived from the third segmentor output and the entropy derived from the fourth segmentor output. In an embodiment, a segmentor such as segmentor 446 and segmentor 436 produces an output for each pixel in a source or target image.

In an embodiment, training of synthesizer 420 is undertaken to reduce the loss that includes one or more loss terms comprising a bidirectional adversarial loss, a segmentation loss, and a cycle consistency loss. In an embodiment, at block 345, a segmentation cross entropy loss term is calculated that quantifies cross entropy loss across predetermined segment classes. In an embodiment, the classes include a set of brain tissue classes including one or more of the border, background, gray matter (GM), cerebrospinal fluid (CSF), and white matter (WM).

In an embodiment, training of synthesizer 420 results in the production of two output synthesizers. At 342, a first generator 422 is produced for synthesizer 420 that produces target domain images from source domain images. At 344, a second generator 424 is produced for synthesizer 420 that produces source domain images from target domain images. In an embodiment, synthesizer 350 receives a first parameter set defining generator 422 through a first data structure that defines the weights and biases for an ANN through a first portion of model 224 determined by synthesizer trainer 221. In an embodiment, synthesizer 350 receives a second parameter set defining generator 424 through a second data structure that defines the weights and biases for an ANN through a second portion of model 224 determined by synthesizer trainer 221.

In an embodiment, an entropy loss also includes a cycle consistency loss term. A cycle consistency loss term is formed, for example, by producing a synthesized target image object 432 and from this producing a pseudo source image object 444. A cycle consistency loss term is then formed by obtaining an entropy calculation based on a first segmentation output of a target image, a second segmentation output of a pseudo target image, a third segmentation output of a source object, and a fourth segmentation output of a pseudo source object. The segmentation loss term is then formed from a sum that includes a norm of a difference between the first segmentation output and the second segmentation output, and a norm of a difference between the third segmentation output and the fourth segmentation output. In an embodiment, the 1-norm is used. In an embodiment, the training of synthesizer 420 is undertaken to reduce the segmentation loss term.

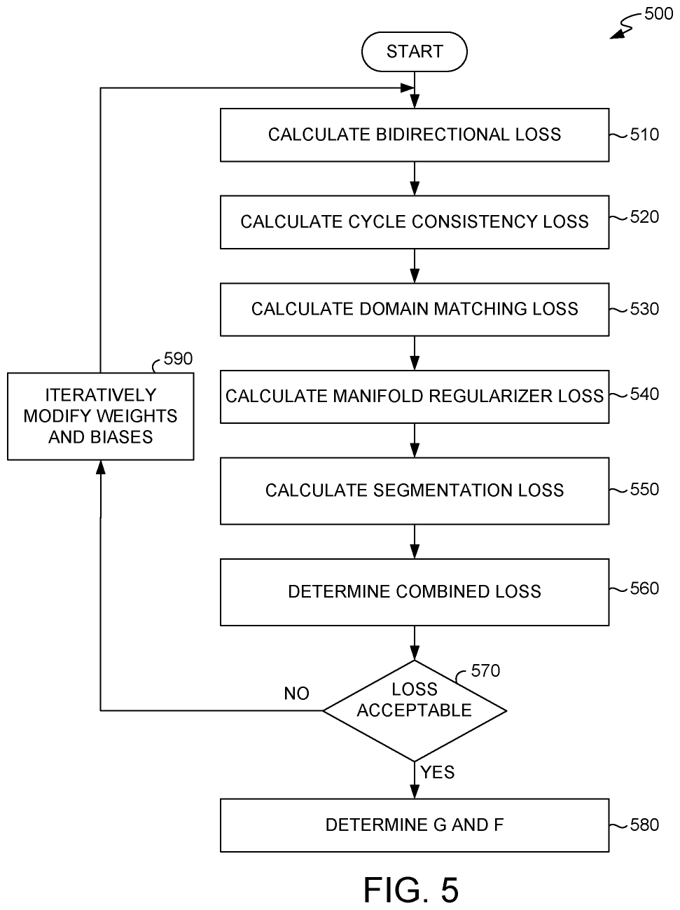

Synthesizer 350 forms an estimate of synthesizer 420, including generator 422 and generator 424 by incorporating information from a source domain corpus and information from a target domain corpus into synthesizer 420 through training. An exemplary method 500 for training synthesizer 420 by synthesizer trainer 221 is shown in block diagram form in FIG. 5. Synthesizer trainer 221 presents to the user a description of a model definition that includes “fully defined, but not trained” together with a graphical control for initiating training. When a user selects the graphical control, preparatory training operations are performed and method 500 is invoked.

Preparatory training operations include, for example, the synthesizer trainer 221 placing model definitions for the selected model into memory, determining fixed components, determining components to be trained, determining batch size, determining a component training sequence (if any), initializing weights and biases into components to be trained, selecting a batch of source samples from the source sample corpus and a batch of target samples from the target sample corpus, and applying selected batches to synthesizer 420. In an embodiment, a learning rate of 0.0002 is set. In an embodiment, different corpus pairs of source corpus and target corpus are identified for each step of training.

In an embodiment, a training sequence includes a descriptor training step, a segmentor training step, and a bidirectional GAN training step. In a descriptor training step descriptors 460 and 470 are trained by an iterative process to approximate the features of the source domain in a descriptor for the source domain and a descriptor for the synthesized source domain, and also to approximate the features of the target domain in a descriptor for the target domain and a descriptor for the synthesized target domain. The descriptors for the source and target domains are then fixed, and the descriptors for the synthesized target domain and synthesized source domain are initialized to be used in the bidirectional GAN training step. In the segmentor training step segmentors 436 and 446 are trained by an iterative process to correctly identify the classes defined. Segmentors 436 and 446 are then fixed for the bidirectional training step. In the bidirectional training step the descriptor of the synthesized sourced domain, the descriptor of the synthesized target domain, generator 422, generator 424, discriminator 452, and discriminator 454 are all identified as components to be trained, and the method of 500 is invoked.

In an embodiment, synthesizer trainer 221 determines that descriptor 470, descriptor 460, generator 422, generator 424, segmentor 436, segmentor 446, discriminator 452 and discriminator 454 are all components to be trained, and no component training sequence is defined, and the method of 500 is invoked.

In an embodiment, to train a synthesizer 420, a common procedure is to take alternating steps of updating generator and discriminator in every batch. The solution may continue along this road and introduce the segmentor updating together with the discriminator first when fixing the generator and other regularizers, and then reverse the process. In an embodiment, the solution may employ the Stochastic Gradient Descent with a mini-batch of size 1 and apply the Adam solver for optimization. Empirically, to control the influence between G and F, the solution may set the balance coefficient δ=10, the trade-off parameter γ=0.3. However, with the 3D generation, a potential problem can be exposed that synthesizing 3D voxels is harder than differentiating between the synthesized result and ground truth, easily leading to the faster learning progress of the discriminator than the generator. The proposed discriminator here cooperates with an additional criterion that both synthesis and segmentation performance may be balanced and satisfied.

Method 500 generally involves calculating one or more loss functions at 510, 520, 530, 540, 550, 560, determining if the loss is acceptable at 570, and if not iterating by returning to the beginning of the iteration loop after modifying weights and biases at 590. When the loss is acceptable generator 422 and generator 424 have been determined at 580. A new batch of source domain samples and a new batch of target domain samples are taken into the iteration loop and applied to system 400 before new loss calculations are performed, e.g. at a return to 510. At 590 the weights and biases of the components being trained are modified in each iteration of the loop to search for an improved set of weights and biases. In an embodiment, the modification is made in accordance with stochastic gradient descent. In an embodiment, the Adam solver is used for training. In an embodiment, the mini-batch size of 1 is used.

At 570 a test is performed to determine if the loss computed is acceptable. In an embodiment, the test simply determines if more iteration loops were planned, if yes, then the test determines that loss is not acceptable and the method proceeds to 590. In an embodiment, the average loss over some number of iterations is calculated, and when the average loss has been approximately equal for some period of time, the loss is determined to be acceptable and the method proceeds to 580.

At 510 a bidirectional dual loss [symbol] _{d} is calculated. At 520 a cycle consistency loss L_{c} is calculated. In an embodiment, a synthesizer 420 is trained by synthesizer trainer 221 to reduce a bidirectional adversarial loss that includes a first visual similarity term comparing a synthesized target image to a target image, and a second visual similarity term comparing a synthesized source image to a source image. In an embodiment, the trained synthesizer 420 is configured in a dual-arranged configuration, as shown in system 400, having generator 422 dual-arranged with generator 424, and corresponding discriminators 452 and 454. In an embodiment, the bidirectional adversarial loss used to train synthesizer 420 includes a bidirectional adversarial loss term and a cycle consistency loss term. In an embodiment, the cycle consistency loss term regularizes mappings in system 400. In an embodiment, the mappings are regularized by comparing distances on a geometric manifold. In an embodiment, the mappings are regularized by comparing feature maps for a set of M selected feature layers. In an embodiment, the M selected feature layers are the deep layers of a deep network. In an embodiment, synthesizer 420 is trained to improve simultaneously a first similarity term comparing a synthesized target object 432 to a target image, and a second similarity term comparing a synthesized source object 442 to a source image.

Conditioned on the result of C, the volumetric cycle-consistency function can be extended to handle the consistent property in both tissue content transformation and anatomical structure segmentation. To enforce a segmentor such as 422 or 424 to be integrated within the cycle transitivity and push C_{X} and C_{Y} to be consistent with each other, [symbol] _{X}˜p_{data(X)}∥C_{X}(X)−C_{X}(F(G(X)))∥1+ [symbol] _{Y}˜p_{data(Y)}∥C_{Y}(Y)−C_{Y}(G(F(Y)))∥1 is added to form a complementary cycle-consistency loss [symbol] _{c}(X, G, C_{X}, γ, F, C_{Y})

At 530 a domain matching loss is calculated. In an embodiment, a combined entropy loss objective is formed that includes loss term computed at 510 that is added to a weighted loss term comprising a domain matching loss calculated at 530. In adjusting weights and biases within synthesizer 420 during training, a combined entropy loss objective is reduced, thus forming a dual-arranged synthesizer 420 including dual-arranged generator 422 and generator 424, and two corresponding discriminators 452 and 454. Thus synthesizer 420 makes use of a domain adapted loss that reduces domain discrepancy between the source domain and the target domain. In an embodiment, synthesizer 420 is trained by synthesizer trainer 221 to translate domain-specific visual features through the use of a multi-kernel maximum mean discrepancy (MK-MMD) criterion. A loss term quantifies MK-MMD which is then reduced upon training by synthesizer trainer 221, resulting in a synthesizer network that translates domain-specific visual features. When an MK-MMD loss term is added to a weighted segmentation loss term computed at 550, and the combined loss is minimized, the synthesizer network is conditioned on a segmentation task performed in the source domain by segmentor 446 and a segmentation task performed in the target domain by segmentor 436.

Although the modeled image distributions over the latent feature space are adopted in the unsupervised cross-modality synthesis problem, an assumption potentially implied is that the representations of both modalities are almost domain invariant. Deep features can disentangle explanatory factors of variations in the data distributions, but the cross-modality distribution discrepancy is still remnant. Motivated by the requirement of boosting unpaired cross-modality data (underlying the same distributions) to be close to each other, especially in the medical images under multiple conditions, the solution may define a jointly-adapted regularization term that intrinsically manifests invariant structures across modalities. To achieve this effect, alternatives employ either Maximum Mean Discrepancy (MMD) or the extended MK-MMD criterion to explore the data statistics of different domains. The jointly-adapted regularizer is proposed to release the assumption of domain invariance. In an embodiment, the solution may make use of the MK-MMD. MK-MMD is employed for the two-sample matching, along with other components of MCMT-GAN, to align the ‘real’ paired data. Specifically, the solution may use the unbiased estimate of MK-MMD to reduce the domain discrepancy, hence this solution is independent of the assumption of ‘same latent variables’. This is done by adding the MK-MMD-based jointly-adapted regularizer to the bidirectional GAN model:

[symbol]

( [symbol] )=∥ [symbol] ₚ_{_{data(X)}}ψ(A^{x})− [symbol] ₚ_{_{data(Y)}}ψ(A^{y})

[symbol]

where [symbol] (·) is interpreted as matching all orders of statistics which can be performed by stochastic gradient descent with the gradient calculated by back-propagation through the generative network, and [symbol] is the characteristic kernel defined on the vectorized element A combining a set of positive definite kernels

[symbol]

$$∈ := \{ {\sum }_{u = 1}^{d} {β}_{u} {k}_{u} : {\sum }_{u = 1}^{d} {β}_{u} = 1 , {β}_{u} \geq 0 , ∀ 𝓊 ∈ \{ 1 , … , d \} \}$$

where

$${\{ {β}_{u} \}}_{u = 1}^{d}$$

is the coefficient for constraining the characteristic of each k_{u·}ψ(·) denotes the nonlinear mapping with

$$k ( {A}^{x} , ) = 〈 ψ ( {A}^{x} ) , ψ ( {A}^{y} ) 〉 = {\{ {A}_{i}^{x} \}}_{i = 1}^{S}$$

and

$$= {\{ \}}_{i = 1}^{T}$$

are the deep features of [symbol] and [symbol] for source and target domains respectively, and [symbol] indicates the reproducing kernel Hilbert space (RKHS) induced by k and ψ. The solution may then integrate the volumetric cycle-consistency and joint adaptation into Eq. (1), yielding the proposed jointly-adapted bidirectional loss:

[symbol]

_{b}(X, [symbol] )= [symbol] _{d}(D_{G},D_{F},G,F)+ [symbol] _{c}(X,G, [symbol] ,F)+ [symbol] ( [symbol] )

At 540 a manifold regularizer loss term is computed. Training of synthesizer 420 is then based on the geometric structure features when the synthesizer trainer 221 trains synthesizer 420 to reduce the manifold regularizer loss term. When synthesizer 420 is fully trained, an additional source object is fed into synthesizer 420 and an additional target object is formed by generator 422 based on the additional source object.

In an embodiment, the solution may use a manifold regularizer. During the learning procedure, high-level features of X and Y are captured through some of the important low-level details of the domain-specific information are lost. Under the limitation of missing visual details, the generations are perceptually meaningful but lacking practical significance especially for medical image analysis. An image manifold may reflect the intrinsic geometric structure underlying the data leading to the generation of appealing results with a realistic overall structure. The solution may, therefore, attempt to preserve the complementary properties by introducing a manifold regularizer (a.k.a. graph Laplacian). Despite the property of maximizing the consistency of intra-domain structure and correlation of inter-domain, such a regularizer is restricted by integrating with deep architecture. To enable graph learning to GAN, meanwhile integrating the aforementioned jointly-adapted regularizer, in an embodiment, the solution may adopt deep features for geometric structure measurement. To be specific, given A^{x} and A^{y} of X and Y respectively, one can construct two q-nearest neighbor graphs [symbol] ^{x} and [symbol] ^{y}, with p vertices. The weight matrices W^{x} and W^{y} of [symbol] ^{x} and [symbol] ^{y} are then defined as W_{i,j}^{X}=1 and W_{i,j}^{Y}=1 if and only if for any two deep features on the manifold with short geodesic distances satisfying: Aᵢ^{x} or Aᵢ^{y}, is among the q-nearest neighbors of Aⱼ^{x} or Aⱼ^{y}; otherwise W_{i,j}^{X}=0 or W_{i,j}^{Y}=0. The domain-specific graph structures are encoded into two weight matrices with the corresponding diagonal matrices Dᵢᵢ^{x}=ΣⱼWᵢⱼ^{X} and Dᵢᵢ^{y}=ΣⱼWᵢⱼ^{Y}, resulting in domain graph Laplacian L^{x}=D^{x}−W^{x} and [symbol] = [symbol] − [symbol] . The solution may then preserve the geometric structures by minimizing the following feature manifold regularization

$$M ( {A}^{x} , {A}^{y} ) = \frac{1}{2} {\sum }_{i , j = 1}^{p} ( {〚 {A}_{i}^{x} - {A}_{j}^{x} 〛}^{2} {W}_{i , j}^{X} + {〚 {A}_{i}^{y} - {A}_{j}^{y} 〛}^{2} {W}_{i , j}^{y} )$$

where M(·) is referred to as the manifold regularizer.

At 550 a segmentation loss is calculated. In an embodiment, an entropy loss term includes a bidirectional adversarial loss term computed at 510 and a segmentation loss term computed at 550. In an embodiment, the segmentation loss term computed at 550 is a cross entropy loss across the segmentation classes. Synthesizer 420 is then trained by synthesizer trainer 221 reducing the entropy loss term, while performing segmentation tasks on source objects with segmentor 446 and performing segmentation tasks on target objects with segmentor 436. The entropy loss term that is reduced is then conditioned on the segmentation tasks performed on the synthesized target image object 432 and segmentation tasks performed on the target image. Likewise, the entropy loss term that is reduced is conditioned on the segmentation tasks performed on the synthesized source image object 442 and segmentation tasks performed on the source image In an embodiment, segmentation information is used for image analysis. In an embodiment, segmentation information is used through semantic segmentation approaches. In an embodiment, Rather than only encouraging the visually-realistic synthesis to approximate the ground truth in an unsupervised bidirectional learning manner, the solution may instead encourage them to have better performance in segmentation as used by the real image. To ensure the synthesized results can satisfy the requirement of later segmentation, and substitute the real acquisitions for medical image analysis, the solution may bridge the gap of task performance between the distributions of generated and real ones. The ideal discriminator has an explicit notion that whether the image is real or synthesized and also match the segmentation performance. More precisely, the responsibility of the proposed discriminator is to judge two major tasks (i.e., the performance of synthesis and segmentation) derived an obvious discriminant: real data & right segmentation. In an embodiment, the task-specific (e.g. text, label, and even image) descriptions and other data may be required for multi-task (especially for synthesis and segmentation) co-evolution coherently. The fault-aware discriminator (involving two branches) is therefore provided to meet the additional condition on segmentation. This is done by feeding the generations to D_{G}, D_{F}, and two cooperative segmentors C_{X}, C_{Y} respectively. C_{X} and C_{Y} are implemented as deconvolution which assigns the tissue label to each voxel of input

$${L}_{s} ( G , {C}_{X} , F , {C}_{Y} ) = {𝔼}_{Y} ∼ {p}_{data} ( Y ) [ - {\sum }_{i = 1} {\sum }_{k = 1} ( {l}_{i}^{k} log ( {C}_{Y} ( {Y}_{i} ) ) + {l}_{i}^{k} log ( {C}_{Y} ( G ( {X}_{i} ) ) ) ) ] {𝔼}_{X} ∼ {p}_{data} ( X ) [ - {\sum }_{i = 1} {\sum }_{k = 1} ( {l}_{i}^{k} log ( {C}_{X} ( {X}_{i} ) ) + {l}_{i}^{k} log ( {C}_{X} ( F ( {Y}_{i} ) ) ) ) ]$$

where [symbol] ₛ is the cross entropy loss, lᵢ^{k} denotes the one-hot encoding corresponding to the i-th sample volume within the k-th tissue class.

At 560 an objective function is determined as a goal of iterative training. In an embodiment, the properties of dual mapping, visual similarity, geometric manifold preserving, and a coherent segmentation together form the whole adversarial loss that enforces associations between similar contents and keeps tissue specificity conditioned on an extra fault (i.e. segmentation performance) of both domains. Correspondingly, the overall loss function for the proposed model can be further updated including the above four parts. The optimization objective then becomes:

$${min}_{G , F , {C}_{X} , {C}_{Y}} {max}_{{D}_{G} , {D}_{F}} {ℒ}_{d} ( {D}_{G} , {D}_{F} , G , F ) + {ℒ}_{s} ( G , {C}_{X} , F , {C}_{Y} ) + {δℒ}_{c} ( X , G , {C}_{X} , y , F , {C}_{Y} ) + γ B ( {A}^{x} , {A}^{y} ) + σ M ( {A}^{x} , {A}^{y} )$$

where δ denotes a balance coefficient for the cycle-consistency loss, γ is the trade-off parameter for the jointly-adapted penalty, and a indicates a weight controlling the manifold regularization. In the above equation, as with the conventional two-player minimax problem, the solution may train the entire model by alternatively maximizing discriminators D_{G}, D_{F} and minimizing a combination of conditional dual mapping loss [symbol] _{d}, cycle-consistency loss [symbol] _{c}, domain RKHS-distance B, and geometry manifold regularization M in the volumetric space

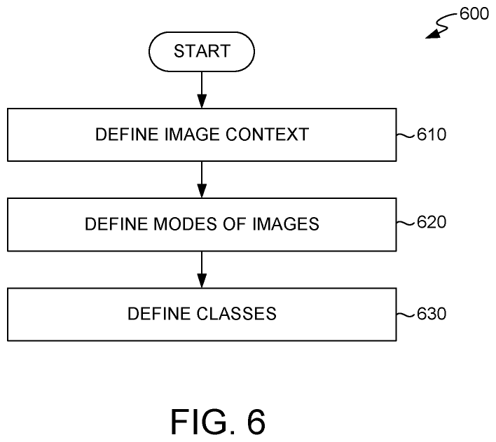

Turning to FIG. 6 there is depicted in 600 a method of defining classes for an application that makes use of a segmentation constraint. In an embodiment, at 610, a category is defined for an image context in which a user desires to synthesize an image. Examples of context include the format of the samples (e.g. 2-D or 3-D), and the application area (e.g. applied in art, medicine, manufacturing, computer-aided design, animation, motion pictures, and computer-aided simulation, etc.). Examples of context also include the scope of the images. For example, 3-D medicine samples might be drawn from a particular portion of anatomy (brain, head, heart, spine, neck, etc.) At 620 the modes of the images to be transformed are defined. For example, in medicine, modes of data collection are identified to represent a different domain of representation that samples might be converted between, (e.g. FLAIR, T1-Weighted, T2-Weighted, PD-weighted, structural MRI, CT). At 630, the classes are defined. Each defined mode is analyzed with data structure analysis to categorize different segments of the images that are desired to be transformed. For example, in brain medical images the classes are determined to be CSF, GM, and WM. In an embodiment, a source and target domain are chosen to model the characteristics a particular type of subject. For example, an adolescent female of 14 years is modeled over a T1-weighted MRI scan as the source domain and with a T2-weighted MRI scan as the target domain by searching database 214 and database 280 for female adolescent scans of both types of scans. A first search step determines a first number of source domain samples available for a type of source domain and a second number of target domain samples available for a type of target domain. A user is presented with the available totals, and the user has the opportunity to narrow or broaden the definition of type.

Experiments were performed to evaluate MCMT-GAN over on two brain datasets: IXI (http://brain-development.org/ixi-dataset/) and NAMIC Multimodality (http://hdl.handle.net/1926/1687) datasets. The IXI dataset involves 578 healthy subjects, each was imaged using a matrix of 256×256×p scanned with either Philips 3T system, Philips 1.5T system, or GE 3T system having 0.94×0.94×1.2 mm3 voxel dimensions. The NAMIC dataset includes 20 subjects (10 normal controls and 10 schizophrenics), each was imaged using a matrix of 128×128×q scanned with a 3T GE system having 1×1×1 mm3 voxel dimensions.

The solution may be evaluated in two scenarios: (1) synthesizing the T2-w images from the PD-w acquisitions and vice versa on the IXI dataset, (2) generating the T1-w images from the T2-w inputs and vice versa on the NAMIC dataset. However, if only a few groups of unpaired data are available on the NAMIC dataset, the images on the IXI dataset may be scaled to 128×128×p voxels for extending the size of training data in this scenario. For quantitative evaluation, one may perform two-fold cross-validation to test this solution. By selecting 230 unpaired Proton Density-weighted (PD-w) and T2-weighted (T2-w) MRI scans from the IXI dataset and 7 unpaired T1-w, T2-w acquisitions form the NAMIC dataset for training, while the remaining data i.e., 118 (IXI) and 6 (NAMIC) for testing. For segmentation, the real scans and the synthesized results may be fed to the model.

The segmentor produces several major brain tissue classes, i.e., Cerebrospinal Fluid (CSF), Gray Matter (GM), and White Matter (WM), yielding the averaged quantification of a whole-brain volume. The tissue prior probability templates used in the segmentor are based on averaging multiple automatically segmented images in standard space of images from either the IXI or the NAMIC dataset, so there is no guarantee that CSF, GM, and WM classes will exactly follow other methods. In addition, a reliable synthesis mechanism is disclosed for generating both visually-realistic and task-effective products.

For the evaluation criteria, one may adopt PSNR, SSIM indices and Dice overlap to objectively assess the quality of the synthesized results and the use of generations on segmentation. Besides the widely used PSNR and SSIM, the Dice overlap is also a well-known volume metric for comparing the quality of two binary label masks. To quantitatively evaluate both visual quality of the synthesized results and the segmentation performance comparing with ground truths, and also to explore the generality of MCMT-GAN, one may test on many tasks using two independent datasets. For brevity, one may refer to different synthesis tasks as (1) PD-w→+T2-w, (2) T2-w→+PD-w, (3) T1-w→+T2-w, (4) T2-w→+T1-w, in which (1-2) are conducted on the IXI dataset corresponded to the first scenario and (34) are explored on the NAMIC dataset corresponded to the second scenario. Both visual and quantitative results are demonstrated. An implemented embodiment consistently yields the best results against prior supervised and unsupervised cross-modality synthesis methods for two datasets.

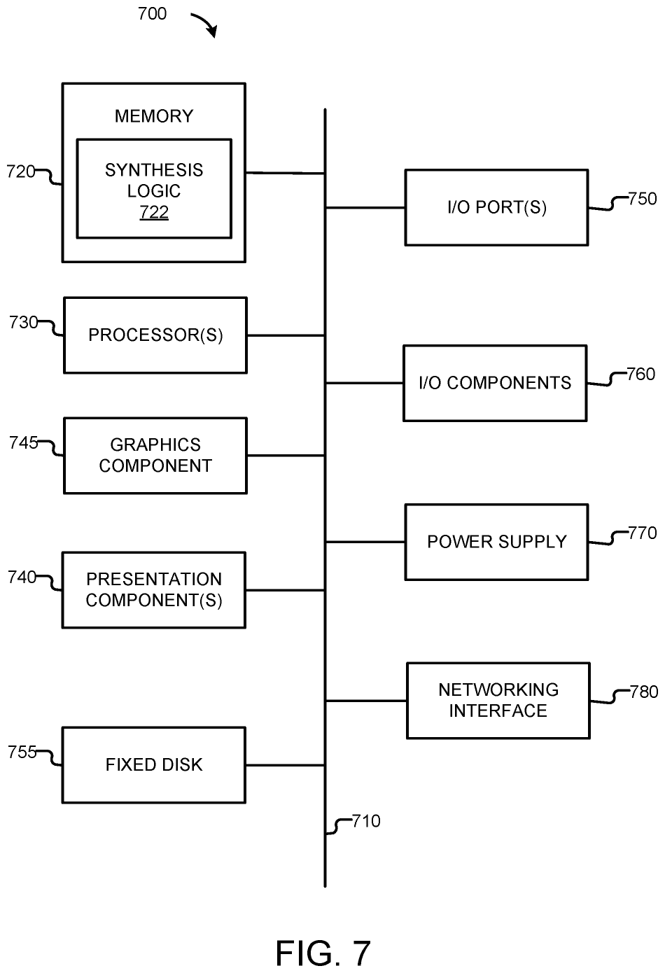

Turning briefly to FIG. 7, there is shown architecture detail of one embodiment of computer 250 that has software instructions for storage of data and programs in computer-readable media. Computing system 700 is representative of a system architecture that is suitable for computer systems such as computer 250 or 290. Components of system 700 are generally coupled together, for example by communication bus 710. One or more CPUs such as processor(s) 730, have internal memory for storage and couple to memory 720 that contains synthesis logic 722, allowing processor(s) 730 to store instructions and data elements in system memory 720, or memory associated with an internal graphics component, which is coupled to presentation component(s) 740 such as one or more graphics displays. In an embodiment, an external graphics component 745 is provided in addition to or in place of a graphics component internal to processor(s) 730 and couples to other components through bus 710. A Bios flash ROM is contained within processor(s) 730. Processor(s) 730 can store instructions and data elements in internal disk storage or in an external fixed disk 755, or make use of I/O port 750 to store on a USB disk, or make use of networking interface 780 for remote storage. User I/O components 760 such as a communication device, a mouse, a touch screen, a joystick, a touch stick, a trackball, or keyboard, coupled to processor(s) 730 through bus 710 as well. The system architecture depicted in FIG. 7 is provided as one example of any number of computer architectures, such as computing architectures that support local, distributed, or cloud-based software platforms, and are suitable for supporting computer 250. In an embodiment, system 700 is implemented as a microsequencer without an arithmetic-logic unit (ALU). In an embodiment, system 700 is implemented as discrete logic that performs the functional equivalent in discrete logic such as a custom controller, a custom chip, Programmable Array Logic (PAL), a Programmable Logic Device (PLD), an Erasable Programmable Logic Device(EPLD), a field-programmable gate array (FPGA) a macrocell array, a complex programmable logic device, a hybrid circuit. Processor(s) 730 are extensible to any I/O device through I/O port(s) 750. The computing system 700 is supplied power by power supply 770. In an embodiment, a graphics processor in graphics component 745 performs computing with software rather than making use of traditional CPUs such as those present in processor(s) 730.

In some embodiments, computing system 700 is a computing system made up of one or more computing devices. Computing system 700 may be a distributed computing system, a data processing system, a centralized computing system, a single computer such as a desktop or laptop computer, or a networked computing system.

Many different arrangements of the various components depicted, as well as components not shown, are possible without departing from the scope of the claims below. Implementations of the disclosure have been described with the intent to be illustrative rather than restrictive. Alternative implementations will become apparent to readers of this disclosure after and because of reading it. Alternative means of implementing the aforementioned can be completed without departing from the scope of the claims below. Certain features and sub-combinations are of utility and may be employed without reference to other features and sub-combinations and are contemplated within the scope of the claims.

For example, in conjunction with specificity requirements and for clarity, the processing performed in a bidirectional GAN was stated in terms of sample, and generally, a voxel was an example of a sample. In some embodiments, a sample is a 2-D image or a 2-D portion of an image.

Additionally, the disclosed methods generally are applicable to unsupervised and unpaired data. In an embodiment, training is performed in a supervised manner. In an embodiment, training is performed using paired data.

Furthermore, in an embodiment, a selection of an input object includes a selection of a frame picture in a 3-D object, such as a 3-D brain scan, and outputting a corresponding picture in a synthesized 3-D object that has been produced by 3D synthesis.

An embodiment stores in library 272 predetermined segmentors 436 and 446 that are fixed throughout adversarial generative training. Texture propagation is performed on each class after segmentation.

In an embodiment, a global registry of models and data is stored in database 280 and available through computer 290 through a data network 230 such as the internet, or the world-wide-web. A user is then able to browse models available in database 280, and to load a model into a synthesizer. A user can perform searches of samples over database 280 and define a corpus over available samples.

In an embodiment, texture details are preserved by copying low-level feature maps from a deep network that represents an input image, and the adaptation system operates by modeling only upper layers. In an embodiment, the lowest two layers are copied. In an embodiment, the lowest three layers are copied.

In an embodiment, a completed model 224 includes segmentor 436 and segmentor 446, generator 422 and generator 424. Thus a model supports embedded segmentation of both source and target images, and supports forward generation through generator 422 or approximate inverse generation through generator 424.

### General Examples

The first general example is an apparatus for synthesizing images. The apparatus comprises a memory having computer programs stored thereon and a processor configured to perform, when executing the computer programs, operations comprising: generating an output target image from an input source image via a first image generator network that is formed based on texture propagation in a bidirectional generative adversarial network that comprises the first image generator network and a second image generator network which is an approximate inverse of the first image generator network, wherein the formation of the first generative network includes processing performed over a corpus of source samples in a source domain and a corpus of target samples in a target domain.

This sub-example may include the subject matter of the first general example and any one of its sub-examples, wherein the texture propagation causes a source image to propagate texture details from a source image to a target image by using feature maps of a deep network acting as a descriptor to preserve local textural details at convolutional layers.

This sub-example may include the subject matter of the first general example and any one of its sub-examples, wherein the feature maps comprise feature maps at a layer L modeling features of a target domain sample which are correlated to feature maps at a layer L modeling features of a synthesized source domain sample and feature maps at a layer L modeling features of a source domain sample which are correlated to the feature maps at a layer L modeling features of a synthesized target domain.

This sub-example may include the subject matter of the first general example and any one of its sub-examples, wherein the first image generator network and the second image generator network are iteratively modified in accordance with an entropy loss that comprises a texture entropy loss term that employs a 1-norm.

This sub-example may include the subject matter of the first general example and any one of its sub-examples, wherein the first image generator network is formed based on a shape prior constraint.

This sub-example may include the subject matter of the first general example and any one of its sub-examples, wherein the shape prior constraint extracts shape information from a target domain sample using a target domain segmentor and extracts shape information from a source domain sample using a source domain segmentor.

This sub-example may include the subject matter of the first general example and any one of its sub-examples, wherein the entropy loss further comprises a segmentation cross entropy loss term.

This sub-example may include the subject matter of the first general example and any one of its sub-examples, wherein the segmentation cross entropy loss term calculates cross entropy loss across a set of brain tissue classes comprising at least one of Cerebrospinal Fluid, Gray Matter and White Matter.

This sub-example may include the subject matter of the first general example and any one of its sub-examples, wherein the entropy loss further comprises at least one of a domain matching loss term, a cycle consistency loss term, and a bidirectional loss term.

This sub-example may include the subject matter of the first general example and any one of its sub-examples, wherein the source domain is a first mode of data collection and the target domain is a second and distinct mode of data collection pertaining to subjects with one or more similar attributes.

This sub-example may include the subject matter of the first general example and any one of its sub-examples, wherein a sample comprises a voxel.

Another example may include one or more non-transitory computer-readable media comprising instructions to cause an apparatus, upon execution of the instructions by one or more processors of the apparatus, to perform any one of the operations associated with the first general example and any one of its sub-examples.

Another example may include an apparatus comprising means to perform any one of the operations associated with the first general example and any one of its sub-examples.

Another example may include a method to perform any one of the operations associated with the first general example and any one of its sub-examples.

The second general example comprises a method for training a first image generator network comprising: receiving a corpus of source samples in a source domain and a corpus of target samples in a target domain, forming a first generator network estimate based on texture propagation through bidirectional generative adversarial network estimation using the information contained in the corpus of source samples and in the corpus of target samples.

This sub-example may include the subject matter of the second general example and any one of its sub-examples, wherein the texture propagation causes a source image to propagate texture details from a source image to a target image by using feature maps of a deep network acting as a descriptor to preserve local textural details at convolutional layers.

This sub-example may include the subject matter of the second general example and any one of its sub-examples, wherein the feature maps comprise feature maps at a layer L modeling features of a target domain sample which are correlated to feature maps at a layer L modeling features of a synthesized source domain sample and feature maps at a layer L modeling features of a source domain sample which are correlated to feature maps at a layer L modeling features of a synthesized target domain sample.

This sub-example may include the subject matter of the second general example and any one of its sub-examples, wherein the first image generator network and the second image generator network are iteratively modified in accordance with an entropy loss that comprises a texture entropy loss term that employs a 1-norm.

This sub-example may include the subject matter of the second general example and any one of its sub-examples, wherein the first image generator network estimate is further based on a shape prior constraint, the shape prior constraint extracts shape information from a target domain sample using a target domain segmentor and extracts shape information from a source domain sample using a source domain segmentor, the entropy loss further comprises a segmentation cross entropy loss term that calculates cross entropy loss across a set of brain tissue classes comprising at least one of cerebrospinal fluid, gray matter and white matter.

This sub-example may include the subject matter of the second general example and any one of its sub-examples, wherein the entropy loss further comprises at least one of a domain matching loss, a cycle consistency loss, and a bidirectional loss.

This sub-example may include the subject matter of the second general example and any one of its sub-examples, wherein the source domain is a first mode of data collection and the target domain is a second and distinct mode of data collection pertaining to subjects with one or more similar attributes.

This sub-example may include the subject matter of the second general example and any one of its sub-examples, wherein a sample comprises a voxel.

Another example may include one or more non-transitory computer-readable media comprising instructions to cause a computer, upon execution of the instructions by one or more processors of the computer, to perform the method of the second general example and any one of its sub-examples.

Another example may include an apparatus comprising means to perform the method of the second general example and any one of its sub-examples.

The third general example is an apparatus for synthesizing images, comprising: a first generator configured to operate on an input source image to produce an output target image, the first generator network having been formed by employing texture propagation and a shape prior constraint through the training of a bidirectional generative adversarial network that comprises the first image generator network and a second image generator network which is an approximate inverse of the first image generator network, wherein the first image generator network and the second image generator network are iteratively modified by processing an input voxel in accordance with an entropy loss that comprises a texture entropy loss term and a segmentation cross entropy loss term.

Another example may include one or more non-transitory computer-readable media comprising instructions to cause an apparatus, upon execution of the instructions by one or more processors of the apparatus, to perform any one of the operations associated with the third general example and any one of its sub-examples.

Another example may include an apparatus comprising means to perform any one of the operations associated with the third general example and any one of its sub-examples.

Another example may include a method to perform any one of the operations associated with the third general example and any one of its sub-examples.

The fourth general example is an apparatus for synthesizing images. The apparatus comprising a memory having computer programs stored thereon and a processor configured to perform, when executing the computer programs, operations comprising: receiving a first source image in a source domain and a first target image in a target domain; training an image synthesizing network with the source image and the target image, the training being based at least in part by generating geometric structure information of the first source image and the first target image, and providing the geometric structure information as two separate inputs to a dual-arranged synthesizer; and synthesizing a second target image from a second source image via the image synthesizing network.

This sub-example may include the subject matter of the fourth general example and any one of its sub-examples, wherein the operations further comprising: reducing a bidirectional adversarial loss for the dual-arranged synthesizer having two dual-arranged generators and two corresponding discriminators, the bidirectional adversarial loss being configured to simultaneously reduce a first visual similarity between a synthesized target image and the target image, and a second visual similarity between a synthesized source image and the source image.

This sub-example may include the subject matter of the fourth general example and any one of its sub-examples, wherein the operations further comprising: reducing a combination of the bidirectional adversarial loss and a domain adapted loss for the image synthesizing network, the domain adapted loss being configured to reduce domain discrepancy between the source domain and the target domain.

This sub-example may include the subject matter of the fourth general example and any one of its sub-examples, wherein the operations further comprising: reducing a combination of the bidirectional adversarial loss and a cycle-consistency loss for the image synthesizing network, the cycle-consistency loss being configured to regularize mappings in the image synthesizing network.

This sub-example may include the subject matter of the fourth general example and any one of its sub-examples, wherein the operations further comprising: generating a first segment based at least in part on the target image; generating a second segment based at least in part on a temporary target image produced via the dual-arranged synthesizer; reducing a combination of the bidirectional adversarial loss and a difference between the first segment and a second segment.

This sub-example may include the subject matter of the fourth general example and any one of its sub-examples, wherein the operations further comprising: wherein the difference between the first segment and a second segment comprises a cross entropy loss based at least in part on classification labels assigned to respective pixels on the temporary target image.

This sub-example may include the subject matter of the fourth general example and any one of its sub-examples, wherein the operations further comprising: producing a temporary target image and a dual generation of the temporary target image via the dual-arranged synthesizer; reducing a difference for a segmentation task performed on the target image and the dual generation of the temporary target image respectively.

This sub-example may include the subject matter of the fourth general example and any one of its sub-examples, wherein the operations further comprising: wherein the image synthesizing network is trained to translate domain-specific visual features, conditioned on a segmentation task, between the first domain and the second domain.

Another example may include one or more non-transitory computer-readable media comprising instructions to cause an apparatus, upon execution of the instructions by one or more processors of the apparatus, to perform any one of the operations associated with the fourth general example and any one of its sub-examples. Another example may include an apparatus comprising means to perform any one of the operations associated with the fourth general example and any one of its sub-examples. Another example may include a method to perform any one of the operations associated with the fourth general example and any one of its sub-examples.

The fifth general example comprises a method for synthesizing images, comprising: identifying domain invariant features between a first source object in a source domain and a first target object in a target domain; determining geometric structure features of the first source object and the first target object based at least in part on the domain invariant features; training a synthesizing network based at least in part on the geometric structure features, and synthesizing, via the synthesizing network, a second target object based at least in part on a second source object.

This sub-example may include the subject matter of the fifth general example and any one of its sub-examples, further comprising: identifying general features from the first source object and the first target object; wherein identifying the domain invariant features comprises identifying the domain invariant features based at least in part on the general features.

This sub-example may include the subject matter of the fifth general example and any one of its sub-examples, wherein determining the geometric structure features comprises learning domain-specific manifold information of the first domain and the second domain.

This sub-example may include the subject matter of the fifth general example and any one of its sub-examples, wherein training the synthesizing network comprises reducing a bidirectional adversarial loss that is configured to simultaneously improve a first similarity between a synthesized target image and the target image, and improve a second similarity between a synthesized source image and the source image.

This sub-example may include the subject matter of the fifth general example and any one of its sub-examples, wherein reducing the bidirectional adversarial loss is further conditioned on a segmentation task performed between the synthesized target image and the target image.

This sub-example may include the subject matter of the fifth general example and any one of its sub-examples, wherein the first source object and the first target object are three-dimensional objects produced under two different imaging modalities of a same physical object.

Another example may include one or more non-transitory computer-readable media comprising instructions to cause a computer, upon execution of the instructions by one or more processors of the computer, to perform the method of the fifth general example and any one of its sub-examples.

Another example may include an apparatus comprising means to perform the method of the fifth general example and any one of its sub-examples.

The sixth general example is a system for synthesizing images, comprising: a user interface to receive a selection of a target domain; and a synthesizer, operatively coupled to the user interface, configured to generate a synthesized object in the target domain from a source object in a source domain, wherein the synthesized object is generated based at least in part on a mapping of features between the source domain and the target domain, the mapping being conditioned on a segmentation task.

This sub-example may include the subject matter of the sixth general example and any one of its sub-examples, further comprising: a feature recognizer configured to identify general features from a training source object in the source domain and a training target object in the target domain; and a domain discrepancy redactor, operatively coupled to the feature recognizer, configured to generate domain invariant features from the general features.

This sub-example may include the subject matter of the sixth general example and any one of its sub-examples, further comprising: a geometric structure preserver, operatively coupled to the domain discrepancy redactor, configured to determine, based at least in part on the domain invariant features, a first plurality of geometric structure features of the training source object and a second plurality of geometric structure features of the training target object.

This sub-example may include the subject matter of the sixth general example and any one of its sub-examples, wherein the synthesizer comprises dual-arranged generators, wherein a first generator is configured to receive the first plurality of geometric structure features via a first pathway, and a second generator is configured to receive the second plurality of geometric structure features via a second pathway, wherein the first generator is configured to generate a first temporary target object based at least in part on the first plurality of geometric structure features, and to generate a second temporary target object based at least in part on a temporary source object generated by the second generator.

This sub-example may include the subject matter of the sixth general example and any one of its sub-examples, wherein the synthesizer comprises dual-arranged discriminators, wherein a first discriminator is configured to improve a first similarity between the training target object and the second temporary target object, and to improve a second similarity between a segment of the target object and a segment of the first temporary target object.

This sub-example may include the subject matter of the sixth general example and any one of its sub-examples, wherein the user interface comprises a first region to display the source object, a second region to display the synthesized object in response to receiving the selection of the target domain via a domain selector displayed on a third region of the user interface.

Another example may include one or more non-transitory computer-readable media comprising instructions to cause a system, upon execution of the instructions by one or more processors of the apparatus, to perform any one of the operations associated with the sixth general example and any one of its sub-examples. Another example may include a system comprising means to perform any one of the operations associated with the sixth general example and any one of its sub-examples. Another example may include a method to perform any one of the operations associated with the sixth general example and any one of its sub-examples. Another example may include a process image synthesis as shown and described herein. Another example may include a system for image synthesis as shown and described herein. Another example may include a device for image synthesis as shown and described herein.

Aspects of the technology described herein provide a system for improved synthesis of a target domain image from a source domain image. A generator that performs the synthesis is formed based on development that effects texture propagation from the first domain to the second domain by making use of a bidirectional generative adversarial network. A framework is provided for training that includes texture propagation with a shape prior constraint.

The foregoing description of one or more implementations provides illustration and description, but is not intended to be exhaustive or to limit the scope of the invention to the precise form disclosed. Modifications and variations are possible in light of the above teachings or may be acquired from practice of various implementations of the invention.

1-20. (canceled)

21. A non-transitory computer-readable storage device encoded with instructions that, when executed, cause one or more processors of a system to perform operations, comprising:
receiving a first source image in a source domain and a first target image in a target domain;
training a 3D image synthesizing network with the first source image and the first target image, the training being based at least in part by generating geometric structure information of the first source image and the first target image, and providing the geometric structure information as two separate inputs to a dual-arranged synthesizer; and
synthesizing a second target image from a second source image via the 3D image synthesizing network.

22. The non-transitory computer-readable storage device of claim 21, wherein the operations further comprising:
reducing a bidirectional adversarial loss for the dual-arranged synthesizer having two dual-arranged generators and two corresponding discriminators, the bidirectional adversarial loss being configured to simultaneously reduce a first visual similarity between a synthesized target image and the first target image, and a second visual similarity between a synthesized source image and the first source image.

23. The non-transitory computer-readable storage device of claim 22, wherein the operations further comprising:
reducing a combination of the bidirectional adversarial loss and a domain adapted loss for the 3D image synthesizing network, the domain adapted loss being configured to reduce domain discrepancy between the source domain and the target domain.

24. The non-transitory computer-readable storage device of claim 22, wherein the operations further comprising:
reducing a combination of the bidirectional adversarial loss and a cycle-consistency loss for the 3D image synthesizing network, the cycle-consistency loss being configured to regularize mappings in the 3D image synthesizing network.

25. The non-transitory computer-readable storage device of claim 22, wherein the operations further comprising:
generating a first segment based at least in part on the first target image;
generating a second segment based at least in part on the synthesized target image produced via the dual-arranged synthesizer;
reducing a combination of the bidirectional adversarial loss and a sum that comprises the first segment and the second segment.

26. The non-transitory computer-readable storage device of claim 25, wherein the sum comprises a cross entropy loss based at least in part on classification labels assigned to respective pixels on the synthesized target image.

27. The non-transitory computer-readable storage device of claim 21, wherein the operations further comprising:
producing a synthesized target image and a pseudo source image from the synthesized target image via the dual-arranged synthesizer;
reducing a difference for a segmentation task performed on the first target image and a pseudo target image that is produced from a synthesized source image.

28. The non-transitory computer-readable storage device of claim 21, wherein the operations further comprising:
training the 3D image synthesizing network to translate domain-specific visual features, conditioned on a segmentation task, between a first domain and a second domain.

29. A computer-implemented method for synthesizing images, comprising:
identifying domain invariant features between a first source object in a source domain and a first target object in a target domain;
determining geometric structure features of the first source object and the first target object based at least in part on the domain invariant features;
training a synthesizing network based at least in part on the geometric structure features; and
synthesizing, via the synthesizing network, a second target object based at least in part on a second source object.

30. The method of claim 29, further comprising:
identifying general features from the first source object and the first target object; wherein identifying the domain invariant features comprises identifying the domain invariant features based at least in part on the general features.

31. The method of claim 29, wherein determining the geometric structure features comprises learning domain-specific manifold information of the source domain and the target domain.

32. The method of claim 29, wherein training the synthesizing network comprises reducing a bidirectional adversarial loss that is configured to simultaneously improve a first similarity between a synthesized target object and the first target object, and improve a second similarity between a synthesized source object and the first source object.

33. The method of claim 32, wherein reducing the bidirectional adversarial loss is further conditioned on a segmentation task performed between the synthesized target object and the first target object.

34. The method of claim 29, wherein the first source object and the first target object are three-dimensional objects produced under two different imaging modalities of a same physical object.

35. A system for synthesizing images, comprising:
a user interface to receive a selection of a target domain; and
a synthesizer, operatively coupled to the user interface, configured to generate a synthesized object in the target domain from a source object in a source domain, wherein the synthesized object is generated based at least in part on a mapping of features between the source domain and the target domain, the mapping being conditioned on a segmentation task.

36. The system of claim 35, further comprising:
a feature recognizer configured to identify general features from a training source object in the source domain and a training target object in the target domain; and
a domain discrepancy reducer, operatively coupled to the feature recognizer, configured to generate domain invariant features from the general features.

37. The system of claim 36, further comprising:
a geometric structure preserver, operatively coupled to the domain discrepancy reducer, configured to determine, based at least in part on the domain invariant features, a first plurality of geometric structure features of the training source object and a second plurality of geometric structure features of the training target object.

38. The system of claim 37, wherein the synthesizer comprises dual-arranged generators, wherein a first generator is configured to receive the first plurality of geometric structure features via a first pathway, and a second generator is configured to receive the second plurality of geometric structure features via a second pathway, wherein the first generator is configured to generate a synthesized target object based at least in part on the first plurality of geometric structure features, and to generate a second temporary target object based at least in part on a synthesized source object generated by the second generator.

39. The system of claim 38, wherein the synthesizer comprises a dual-arranged discriminators, wherein a first discriminator is configured to improve a first similarity between the training target object and a second pseudo target object, and to improve a second similarity between a first segment of the training target object and a second segment of a first temporary target object.

40. The system of claim 35, wherein the user interface comprises a first region to display the source object, a second region to display the synthesized object in response to receiving the selection of the target domain via a domain selector displayed on a third region of the user interface.
